(19) United States
(12) Patent Application Publication
(10) Pub. No.: US 2023/0320974 A1
(43) Pub. Date: Oct. 12, 2023

(54) Systems and Methods for Altering Hair Texture

(71) Applicant: Tanisha Knox, Los Angeles, CA (US)

(72) Inventor: Tanisha Knox, Los Angeles, CA (US)

(21) Appl. No.: 18/042,217

(22) PCT Filed: Aug. 23, 2021
(86) PCT No.: PCT/US2021/047099

§ 371 Date: Feb. 19, 2023

Related U.S. Application Data
(60) Provisional application No. 63/069,231, filed on Aug. 24, 2020.

(30) Foreign Application Priority Data
Aug. 23, 2021 (WO) PCT/US2021/047099

(51) Int. Cl. A61K 8/92; A61K 8/19; A61K 8/9789; A61K 8/9794; A61K 8/64; A61Q 5/02; A61Q 5/06; A61Q 5/12

(52) U.S. Cl. CPC A61K 8/922; A61K 8/19; A61K 8/9789; A61K 8/9794; A61K 8/645; A61Q 5/02; A61Q 5/06; A61Q 5/12; A61K 2800/30; A61K 2800/34; A61K 2800/884

(57) ABSTRACT

The present disclosure relates to air texture altering systems and methods that, among other things, loosen the curl pattern of the hair without straightening the hair using oils, other natural ingredients, and low concentrations of sodium hydroxide, in a manner that reduces the use of harsh and/or synthetic chemicals.

### CROSS-REFERENCE TO RELATED APPLICATIONS

This application is a national stage application, filed under 35 U.S.C. § 371, of International Patent Application No. PCT/US2021/47099, filed on Aug. 23, 2021, which claims the benefit of U.S. Provisional Patent Application No. 63/069,231, filed on Aug. 24, 2020, each of which are incorporated herein by reference in its entirety.

### FEDERALLY SPONSORED RESEARCH AND DEVELOPMENT

Not applicable.

### BACKGROUND OF THE INVENTION

### Field of the Invention.

Embodiments herein relate generally to systems and methods for altering hair texture and, more specifically, to systems and methods for altering the curl pattern of hair without straightening the hair.

### Description of Arguably Related Art Including Information Disclosed for 37 CFR 1.97 and 1.98

U.S. Published Patent Application No. US2009/0165812 filed by Resnick et al. “Resnick” discloses a hair texture altering system, the hair texturing system comprising: a texture altering mixture. Further provided are one or more hair straightening system comprising (a) the hair straightening formulations set forth above; (b) one or more shampoos, and (c) one or more conditioners; a protective mixture. The systems include a skin protectant. For the scalp protectant, coat and gently rub the scalp with a thin film of cellulose sulfate gel using a small hand-held applicator or fingers. The cellulose sulfate is applied as a gel (alkaline pH at 9-10) to the scalp concentrations ranging from 0.6%-5% with a preferred concentration of 2-3%; a porosity mixture, wherein the porosity mixture is formulated to prepare the hair for receiving the texture altering mixture. The systems include a hair relaxer. This product will swell and open the cuticle and make it semi-porous due to the breakage of the disulfide bond; a shampoo. The systems include a hair relaxer (e.g., shampoo). A shampoo that is slightly basic (pH 7-8) and containing citric acid (buffered with sodium citrate) can be used to relax the hair; and a conditioning mixture, wherein the conditioning mixture is configured to improve a condition of the hair. The systems include a hair fixative. The hair fixative preparation contains hydrolyzed keratin and epigallocatechin gallate as the other main active ingredients. It has been found that the use of particular natural compound in predetermined proportions achieves a straightening effect while simultaneously conditioning the hair.

U.S. Published Patent Application NO. US2004/0110650 filed by Siddiqui et al. (“Siddiqui”) discloses a hair texture altering system, the hair texture system comprising: a texture altering mixture (shampoo and conditioner); a shampoo mixture, wherein the formulation is included in separate hair care products (such as a shampoo and a conditioner); and a conditioning mixture, wherein the conditioning mixture is configured to improve a condition of the hair.

U.S. Pat. No. 5,060,680 issued to Akhtar discloses a method of altering hair texture comprising the steps of: applying a texture altering mixture to hair. Auxiliary amounts of the texturing and strengthening agent can be applied to the virgin outgrowth before applying the hair straightener. Employing a shampoo is usually preferred for purposes of cleansing and removing from the hair residual hair straightener.

A need exists for systems and methods for altering hair texture without the use of free of parabens, sulfates, silicones, synthetic fragrance, mineral oil, petrolatum, and synthetic petrochemicals.

A need exists for systems and methods for altering hair texture by reforming the curl pattern rather than straightening the curl pattern.

A need exists for systems and methods for altering hair texture by reforming the curl pattern rather than straightening the curl pattern by add moisture to the hair using one or more natural oils.

None of the patent references disclose the invention.

### SUMMARY OF THE INVENTION

It is understood that the present disclosure is not limited in its application to the details of construction or to the arrangement of the components described in the following illustrated drawing or in the detailed description.

Provided herein are systems and methods for altering hair texture and, more specifically, systems and methods for altering the curl pattern of hair without straightening the hair in a manner that reduces the use of harmful and/or synthetic materials while using natural and/or plant-based or plant-derived ingredients that improve the condition of the hair and reduce the possibility of damage to the hair. A hair texture altering system of an embodiment of the present disclosure comprises a texture altering mixture, wherein the texture altering mixture is formulated to reform, but not straighten, a curl pattern of hair, for example curly hair, super curly hair, or wavy hair. The texture altering system may also add moisture to the hair using one or more natural oils, which may include about 0.10% by weight simmondsia chinensis (jojoba) seed oil and/or about 0.10% by weight helianthus annuus (sunflower) seed oil. The texture altering mixture may include about 1.35% by weight sodium hydroxide, about 1.48% by weight of sodium hydroxide, or about 1.35% to 1.75% by weight sodium hydroxide. The texture altering mixture may also be free of parabens, sulfates, silicones, synthetic fragrance, mineral oil, petrolatum, and synthetic petrochemicals.

The texture altering system of an embodiment of the present disclosure also includes a protective mixture, wherein the protective mixture is formulated to form a barrier between the texture altering mixture and a scalp portion of a head located at the roof of the hair. The protective mixture may include one or more natural oils, for example, simmondsia chinensis (jojoba) seed oil. The protective mixture may include a base ingredient, where the base ingredient includes at least one or more natural oils, and where at least one of such natural oils may include about 55.0% by weight helianthus annuus (sunflower) seed oil. The protective mixture may further include curcuma longa (turmeric) root extract. The protective mixture may have a consistency of a gel-like oil, and may be free of parabens, sulfates, silicones, synthetic fragrance, mineral oil, petrolatum, and synthetic petrochemicals.

The texture altering system of an embodiment of the present disclosure also includes a porosity mixture, wherein the porosity mixture is formulated to prepare the hair for receiving the texture altering mixture by coating the hair with proteins and/or amino acids for even distribution and absorption of the texture altering mixture on the hair.

The texture altering system of an embodiment of the present disclosure further includes a neutralizing shampoo mixture, where the neutralizing shampoo mixture is formulated to neutralize the texture altering mixture and add moisture to the hair. The neutralizing shampoo mixture may include 0.10% by weight of aloe barbadensis leaf juice to add moisture to the hair. And the neutralizing shampoo may be free of parabens, sulfates, silicones, synthetic fragrance, mineral oil, petrolatum, and synthetic petrochemicals.

The texture altering system of an embodiment of the present disclosure also includes a moisturizing shampoo mixture, where the moisturizing shampoo mixture is formulated to moisturize the hair using, for example, about 0.10% by weight of aloe barbadensis leaf juice to moisturize the hair.

The texture altering system of an embodiment of the present disclosure further includes a conditioning mixture, where the conditioning mixture is configured to improve a condition of the hair by moisturizing the hair using one or more natural oils, and where at least one of the one or more natural oils is selected from a group consisting of about 1.50% by weight of simmondsia chinensis (jojoba) seed oil and about 1.50% by weight of persea gratissima (avocado) oil, guar hydroxypropyltrimonium chloride, and Oryza sativa (rice) bran extract. The conditioning mixture may also strengthen the hair using one or more proteins to strengthen the hair, and where at least one of the one or more proteins is selected from a group consisting of hydrolyzed wheat protein, hydrolyzed soy protein, or hydrolyzed corn protein. The conditioning mixture may also be free of parabens, sulfates, silicones, synthetic fragrance, mineral oil, petrolatum, and synthetic petrochemicals.

The present disclosure further includes a method of altering hair texture comprising the steps of: applying a texture altering mixture to hair, wherein the texture altering mixture is formulated to reform, but not straighten, a curl pattern of the hair; applying a protective mixture, wherein the protective mixture is formulated to create a barrier between the texture altering mixture and a scalp portion of a head located at the root of the hair; applying a porosity mixture to the hair, wherein the porosity mixture is formulated to prepare the hair for application of the texture altering mixture; applying a neutralizing shampoo mixture to the hair, wherein the neutralizing shampoo mixture is formulated to neutralize the texture altering mixture and add moisture to the hair; applying a moisturizing shampoo mixture to the hair, wherein the moisturizing shampoo mixture is formulated to moisturize the hair; and applying a conditioning mixture, wherein the conditioning mixture is configured to improve the condition of the hair, and wherein improving the condition of the hair comprises moisturizing and strengthening the hair. The texture altering mixture of the method may include about 1.35% by weight sodium hydroxide or about 1.48% by weight sodium hydroxide.

It is an object of this invention to provide systems and methods for altering hair texture without the use of free of parabens, sulfates, silicones, synthetic fragrance, mineral oil, petrolatum, and synthetic petrochemicals.

It is yet another object of this invention to provide systems and methods for altering hair texture by reforming the curl pattern rather than straightening the curl pattern.

It is an object of the invention to provide systems and methods for altering hair texture by reforming the curl pattern rather than straightening the curl pattern by add moisture to the hair using one or more natural oils.

These and other aspects, objects, embodiments, and advantages of the invention will become apparent from the accompanying drawing and the following detailed description of the preferred embodiments of the invention.

### BRIEF DESCRIPTION OF THE DRAWING

The invention may be more readily described by reference to the accompanying drawing figures and the following description of the drawing figures. The reference numbers apply to each embodiment of the invention. In the drawing:

FIG. 1 illustrates an exemplary hair texture altering system consistent with an embodiment of the present disclosure.

FIG. 2 illustrates an exemplary method for altering hair texture consistent with an embodiment of the present disclosure.

### DETAILED DESCRIPTION OF THE INVENTION

A detailed description of the embodiments of the present disclosure is provided below. While several embodiments are described, the disclosure is not limited to any one embodiment, but instead encompasses numerous alternatives, modifications, and equivalents. In addition, while numerous specific details are set forth in the following description to provide a thorough understanding of the embodiments disclosed herein, some embodiments can be practiced without some or all of these details. Moreover, for clarity, certain technical material that is known in the related art has not been described in detail to avoid unnecessarily obscuring the disclosure.

The description may use the terms “embodiment,” or “embodiments,” which may each refer to one or more of the same or different embodiments. The terms “comprising,” “including,” “having,” and the like, as used with respect to embodiments, are synonymous, and are generally intended as “open” terms, for example, the term “includes” should be interpreted as “includes but is not limited to,” the term “including” should be interpreted as “including but not limited to,” and the term “having” should be interpreted as “having at least.”

Regarding the use of any plural and/or singular terms herein, those of skill in the relevant art can translate from the plural to the singular and/or from the singular to the plural as is appropriate to the context and/or application. The various singular and/or plural permutations may be expressly set forth herein for the sake of clarity.

As used in this description, the term “a” or “an” means “at least one” or “one or more.” The singular forms “a,” “an,” and “the” include plural references unless the context clearly dictates otherwise. Any reference to “or” herein is intended to encompass “and/or” unless otherwise stated. The term “about” or “approximately” refers to a range of values within plus or minus 10% of the specified number.

Unless otherwise defined, all technical terms used herein have the same meaning as commonly understood by one of ordinary skill in the art to which this disclosure belongs.

The embodiments of the disclosure may be understood by reference to the drawing figures, wherein like parts may be designated by like numerals. The components of the disclosed embodiments, as generally described and illustrated in the figures herein, could be arranged and designed in a variety of different configurations. Thus, the following detailed description of the embodiments of the disclosure is not intended to limit the scope of the disclosure, as claimed, but is merely representative of possible embodiments of the disclosure. In addition, the steps of any method disclosed herein may include variations, or even variations sequentially, and the step may be repeated, unless otherwise specified.

Generally, human hair texture is determined by shape. As hair grows out from the scalp, it assumes the shape of the follicle. The flatter the cross-section of a strand of hair, the curlier the hair. The more circular the cross-section of a strand of hair, the straighter the hair. The more oval-shaped the cross-section of a strand of hair, the wavier the hair. Additionally, for everyone, each strand of hair is formed of proteins called keratin. There are three types of chemical bonds that attract the keratin proteins to each other and are responsible for the strength and elasticity of human hair—namely, hydrogen, salt, and disulfide bonds. Disulfide bonds, in particular, add strength to the keratin protein. Thus, disulfide bonds must be altered to change the shape of the hair.

A common approach to reshaping curlier hair involves the use of chemical straighteners such as hair relaxers and other similar products. These products typically contain harsh and/or noxious chemicals such as formaldehyde, glutaraldehyde and the like, or high concentrations of sodium hydroxide (about 2.0% to 2.5% by weight), which are designed to break the disulfide bonds in the hair to straighten the hair. This process, however, may leave the hair straightened, weak, brittle, and prone to breakage. Such products may even burn the skin, cause permanent damage to the scalp, and lead to hair loss. Moreover, such products may contain petroleum-based ingredients, mineral oil, animal-derived ingredients, and synthetic ingredients, each of which may be harmful.

Thus, various embodiments of the present disclosure herein include system and methods that, among other things, break the disulfide bonds of the hair to loosen the curl pattern of the hair without straightening the hair by using oils, other natural ingredients, and lower concentrations of sodium hydroxide. Additionally, such embodiments reduce the use of harsh and/or synthetic chemicals like those found in most chemical relaxers or similar products.

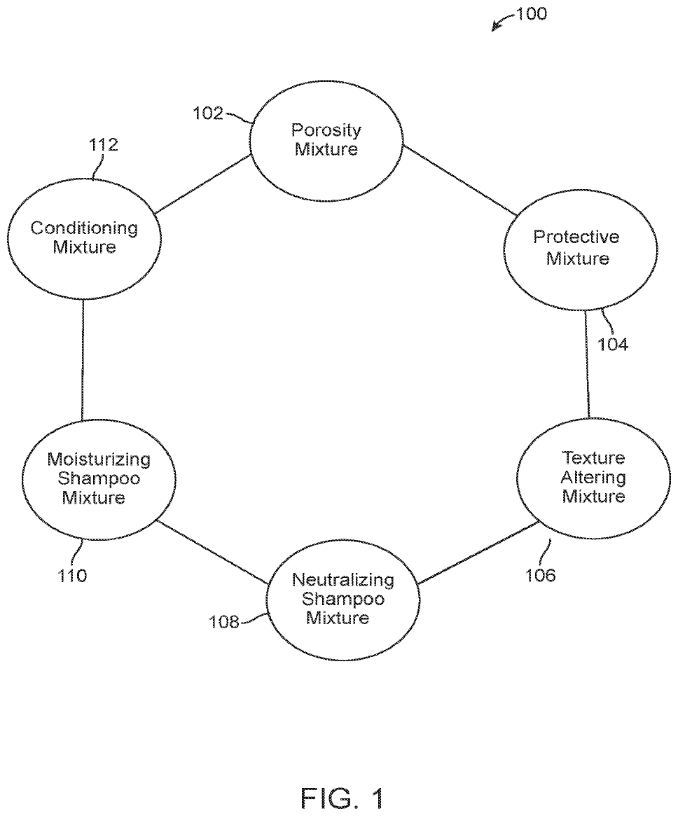

In accordance with the various embodiments herein, an exemplary hair texture altering system 100 is shown in FIG. 1. In one embodiment, the texture altering system 100 may comprising a porosity mixture 102, a protective mixture 104, a texture altering mixture 106, a neutralizing shampoo mixture 108, a moisturizing shampoo mixture 110, and a conditioning mixture 112.

Consistent with embodiments herein, each of the mixtures 102-112 of the hair texture altering system 100 may be formulated to be free of parabens, sulfates, silicones, synthetic fragrance, harmful ingredients, as well as scalp irritating ingredients that may cause a dry and/or flaxy scalp. Each of the mixtures 102-112 of the hair texture altering system 100 may also be formulated using a majority natural, plant, and/or plant-derived ingredients, including natural and essential oils, and the like. Additionally, each of the mixtures 102-112 may comprise a PETA Certification, for example, a vegan and cruelty-free certification, and be free from animal testing.

### Porosity

In one embodiment, the porosity mixture 102 of the system 100 may prepare the hair for application of the texture altering mixture 106 (discussed below) by coating the hair with protein and amino acids or the like to fill in any pores, such as open spaces, that may exist in the hair cuticle. This coating allows for even distribution and absorption of the texture altering mixture 106 to prevent overly porous hair from absorbing the texture altering mixture 106 unevenly and/or faster than desired.

The porosity mixture 102 ay be applied to the shaft and the ends of the hair while dry. No rinsing is required. In one embodiment, the porosity mixture 102 may be formulated as a liquid that can be sprayed on the hair; however, other formulations of the porosity mixture 102 are possible without departing from embodiments of the present invention.

In one embodiment, the porosity mixture 102 may comprised the ingredients shown in Table 1. All percentages in Table 1 are by weight and approximate for the entire composition, making up 100% as shown in Table 1.

TABLE 1
Porosity Mixture Example

| INCI Name | Purpose | Percentage |
|---|---|---|
| Water | | 76.78 |
| Propanediol | Humectant | 10.00 |
| Tetrasodium Glutamate | Chelator | 0.20 |
| Diacetate | | |
| Sodium Citrate | pH Adjuster | 0.10 |
| Citric Acid | pH Adjuster | 0.22 |
| Water (and) Wheat | Active - Hair | 6.00 |
| Amino Acids (and) Soy | Conditioning | |
| Amino Acids (and) | | |
| Arginine HCl (and) | | |
| Serine (and) Threonine | | |
| Water (and) Hydrolyzed | Active - Hair | 3.00 |
| Wheat Protein (and) | Conditioning | |
| Hydrolyzed Soy Protein | | |
| (and) Hydrolyzed Corn | | |
| Protein | | |
| Polyquaternium-73 | Active - Hair | 2.00 |
| | Conditioning | |
| Cetrimonium Chloride | Active - Hair | 0.20 |
| | Conditioning | |
| Sodium Benzoate (and) | Preservative | 1.00 |
| Potassium Sorbate (and) | | |
| Water | | |
| Panthenol | Active - Hair | 0.50 |
| | Conditioning | |
| | | 100% |

### Protection

Referring still to FIG. 1, the protective mixture 104 of the system 100 may protect the scalp during application of the texture altering mixture 106 by creating a barrier between the skin and the texture altering mixture 106. In one embodiment, by applying the protective mixture 104 directly to the scalp as a pre-treatment prior to applying the texture altering mixture 106 to the hair, the possibility of skin irritation is reduced.

In one embodiment, the protective mixture 104 may comprise various natural and/or essential oil ingredients, which give the protective mixture 104 a gel-like oil or “gel-oil” consistency. By using natural and/or essential oils to formulate the protective mixture 104, potentially harmful ingredients such as mineral oils, petroleum-based ingredients, synthetic ingredients, and the like, can be avoided.

For example, consistent with embodiments of the present disclosure, a base, namely, a principle ingredient, of the protective mixture 104 may be formulated by using one or more natural and/or essential oils. In one embodiment, as shown in Table 2 below, a base of about 55% by weight helianthus annuus seed oil (sunflower oil) may be used, although other natural oils such as coconut oil, and the like, are possible. In another example, one or more essential oils may be used to formulate the protective mixture 104 for fragrance. For example, in one embodiment, as shown in Table 2, the following essential oils may be combined to form a fragrance of the protective mixture 104: thuja occidentalis leaf (white cedar) oil, ormenis multicaulis (chamomile) oil, eugenia caryophyllus (clove) leaf oil, Cymbopogon Martini oil, pelargonium graveolens flower (geranium) oil, and fusanus spicatus wood (sandal wood) oil. However, other essential oils may be used alone or in combination to form a fragrance of the protective mixture 104. Using essential oil(s) to form the protective mixture 104 fragrance alleviates the use of synthetic or other chemical ingredients that are typically used to create fragrance in hair products.

In one embodiment, the protective mixture 104 may comprise the ingredients shown in Table 2 below. All percentages in Table 2 are by weight and approximate for the entire composition, making up 100% as shown in Table 2.

TABLE 2
Protective Mixture Example

| INCI Name | Purpose | Percentage |
|---|---|---|
| Helianthus Annuus (Sunflower) Seed Oil | Emollient | 55.00 |
| Tribehenin | Thickener | 6.00 |
| Glyceryl Myristate (and) Glyceryl Palmitate | Thickener | 4.50 |
| (and) Glyceryl Stearate (and) Glyceryl | | |
| Ricinoleate (and) Glyceryl Eicosanedioate | | |
| (and) Dextrin Palmitate | | |
| Oryza Sativa (Rice) Bran Extract (and) | Antioxidant | 1.00 |
| Rosmarinus Officinalis (Rosemary) Extract | | |
| (and) Helianthus Annuus (Sunflower) | | |
| Extract (and) Tocopherol | | |
| Butyrospermum Parkii (Shea) Butter (and) | Active | 4.00 |
| Elaeis Guineensis (Palm) Butter (and) | | |
| Cocos Nucifera (Coconut) Oil (and) | | |
| Simmondsia Chinensis (Jojoba) Seed Oil | | |
| (and) Curcuma Longa (Turmeric) Root Extract | | |
| (and) Bisabolol | | |
| Helianthus Annuus (Sunflower) Seed Oil | Emollient | 28.60 |
| Simmondsia Chinensis (Jojoba) Seed Oil | Emollient | 0.10 |
| Rosa Damascena Flower Extract (and) | Fragrance | 0.80 |
| Thuja Occidentalis (White Cedar) Leaf Oil | | |
| (and) Bulnesia Sarmientoi Wood Oil (and) | | |
| Ormenis Multicaulis (Chamomile) Oil (and) | | |
| Eugenia Caryophyllus (Clove) Leaf Oil | | |
| (and) Cymbopogon Martini Oil (and) | | |
| Pelargonium Graveolens Flower (Geranium) | | |
| Oil (and) Fusanus Spicatus Wood (Sandal | | |
| Wood) Oil | | |
| | | 100% |

### Texture Altering

Referring again to FIG. 1, as discussed above, each strand of hair is composed of chemical bonds call hydrogen, salt, and disulfide bonds. The more disulfide that occurs in the hair fiber, the curlier the hair. In one embodiment, the texture altering mixture 106 of the system 100 may break the disulfide bonds of the hair causing the natural curl pattern of the hair shaft to reform into a less curly, wavy, or smoother curl pattern without straightening the hair. As a result, the hair may be more easily managed, including for hair having a tightly coiled or super curly curl pattern.

In one embodiment, the texture altering mixture 106 may be a mostly plant-based and/or plant-derived formulation that comprises a small amount of sodium hydroxide, for example about 1.25% to 1.75%. The texture altering mixture 106 may be formulated into a light cream (or equivalent) and applied to the hair's root, shaft, and ends for a predetermined period of time, for example, between 20 to 60 minutes, depending upon the desired curl pattern. The amount of sodium hydroxide present in the texture altering mixture 106 may also be increased or decreased, depending upon the desired curl pattern. For example, in one embodiment, 1.35% by weight sodium hydroxide may be used to slightly loosen the curl pattern of the hair, and in another embodiment, 1.48% by weight sodium hydroxide may be used to loosen the curl pattern even further, and so on.

Upon the expiration of the pre-determined time, as discussed in further detail below, the texture altering mixture 106 may be rinsed from the hair with warm, for example, approximately 32° C. to 43° C., water and neutralized using a neutralizing mixture 108 (discussed below). Approximately six to eight weeks after the initial (and each subsequent) application of the texture altering mixture 106, the system 100 may be used to address any new hair growth.

In one embodiment, the texture altering mixture 106 may comprise a mixture of ingredients shown in Table 3 below. All percentages in Table 3 are by weight and approximate for the entire mixture, making up 100% as shown in Table 3.

TABLE 3
Texture Altering Mixture Example

| INCI Name | Purpose | Percentage |
|---|---|---|
| Water | | 65.02 |
| Sodium Hydroxide | pH Adjuster | 1.48 |
| Behentrimonium Chloride | Active - Hair | 0.60 |
| | Conditioning | |
| Polysorbate 60 (and) | Emulsifier | 5.50 |
| Cetearyl Alcohol | | |
| Ceteareth-20 (and) | Emulsifier | 5.20 |
| Cetearyl Alcohol | | |
| C13-15 Alkane | Emollient | 14.00 |
| Simmondsia Chinensis | Emollient | 0.10 |
| (Jojoba) Seed Oil | | |
| Helianthus Annuus | Emollient | 0.10 |
| (Sunflower) Seed Oil | | |
| Propanediol | Humectant | 8.00 |
| | | 100% |

### Neutralizing

As a result of applying the texture altering mixture 106 to the hair, the hair's pH level may increase from normal (e.g., 4.5 to 5.5) or neutral (e.g., 7) levels up to 13. The neutralizing shampoo mixture 108 of the system 100 may be used following application of the texture altering mixture 106 to, among other things, gently cleanse the hair and lower the pH of the hair to normal levels and to moisturize the hair. Lowering the hair to normal or neutral pH levels seals the hair cuticle, locks in natural hair oils, prevents unwanted hair breakage, and promotes hair growth.

In addition to returning the hair back to its normal pH level, certain ingredients of the neutralizing shampoo mixture 108 may provide moisture and add smoothness to the hair without the use of silicones, sulfates, or parabens. For example, in one embodiment, as shown in Table 4, guar hydroxypropyltrimonium chloride, aloe barbadensis leaf juice, and the like, may be used to add moisture to the hair.

In one embodiment, the neutralizing shampoo mixture 108 may comprise the ingredients shown in Table 4 below. All percentages in Table 4 are approximate by weight for the entire mixture, making up 100% as shown in Table 4.

TABLE 4
Neutralizing Shampoo Mixture Example

| INCI Name | Purpose | Percentage |
|---|---|---|
| Water | | 59.90 |
| Phenoxyethanol (and) | Preservative | 0.80 |
| Caprylyl Glycol (and) | | |
| Ethylhexylglycerin (and) | | |
| Hexylene Glycol | | |
| Tetrasodium Glutamate | Chelator | 0.20 |
| Diacetate | | |
| Guar Hydroxypropyltrimonium | Active - Hair | 0.25 |
| Chloride | Conditioning | |
| Citric Acid | pH Adjuster | 0.50 |
| Water (and) Sodium | Cleansing | 26.00 |
| Lauroyl Methyl Isethionate | Surfactant | |
| (and) Cocamidopropyl Betaine | | |
| (and) Sodium Cocoyl Isethionate | | |
| (and) Sodium Methyl Oleoyl Taurate | | |
| Cocamidopropyl Betaine | Cleansing | 6.00 |
| | Surfactant | |
| Aloe Barbadensis Leaf Juice | Active | 0.10 |
| Polyquaternium-73 | Active - Hair | 1.60 |
| | Conditioning | |
| Polyquaternium-7 | Active - Hair | 1.20 |
| | Conditioning | |
| Aqua (and) (Wheat Amino Acids | Active - Hair | 0.10 |
| (and) Soy Amino Acids (and) | Conditioning | |
| Arginine HCl (and) Serine (and) | | |
| Threonine | | |
| Sodium Benzoate | Preservative | 0.20 |
| Phenolsulfonphthalein | Color Indicator | 0.002 |
| Caprylyl/Capryl Glucoside (and) | Solubilizer | 1.50 |
| Water (and) Sodium Cocoyl Glutamate | | |
| (and) Glyceryl Caprylate (and) | | |
| Citric Acid (and) Polyglyceryl-6 | | |
| Oleate (and) Sodium Surfactin | | |
| Sorbeth-230 Tetraoleate (and) | Thickener | 0.80 |
| Decyl Glucoside (and) Sorbitan Laurate | | |
| Rosa Damascena Flower Extract (and) | Fragrance | 0.85 |
| Thuja Occidentalis (White Cedar) Leaf | | |
| Oil (and) Eugenia Caryophyllus (Clove) | | |
| Leaf Oil (and) Cymbopogon Martini Oil | | |
| (and) Pelargonium Graveolens Flower | | |
| (Geranium) Oil (and) Fusanus Spicatus | | |
| Wood (Sandal Wood) Oil | | |
| | | 100% |

In one embodiment, the neutralizing shampoo mixture 108 may comprise an indicator that may be activated when residual texture altering mixture 106 is detected in the hair. For example, after rinsing the texture altering mixture 106 from the hair, the neutralizing shampoo mixture 108 may be applied to the hair and worked into a lather. In one embodiment, a color indicator may be activated when residual texture altering mixture 106 is detected in the hair, which may cause the neutralizing shampoo mixture 108 lather to turn a particular color (e.g., pink, etc) to alert a stylist or another that additional shampooing using the neutralizing shampoo mixture 108 is required to remove all of the texture altering mixture 106 from the hair. In one embodiment, as shown in Table 4 above, an indicator may comprise phenolsulfonphthalein; however, other indicators are possible consistent with embodiments of the present disclosure.

When all of the texture altering mixture 106 is removed from the hair, the neutralizing shampoo mixture 108 lather may be white or clear. Removing all of the texture altering mixture 106 from the hair may prevent hair breakage or other damage to the hair.

### Moisturizing

Still referring to FIG. 1, the moisturizing shampoo mixture 110 of the system 100 may add moisture back to the hair leaving it hydrated and shiny without the use of silicones, sulfates, or parabens. The moisturizing shampoo mixture 110 may be applied to the hair following application of the neutralizing shampoo mixture 108. The moisturizing shampoo 110 may also remove any residual texture altering mixture 106 from the hair that was not removed by the neutralizing shampoo mixture 108. In one embodiment, the moisturizing shampoo mixture 110 may include a pH adjuster to assist in returning the hair to normal pH levels following application of the texture altering mixture 106.

In one embodiment, the moisturizing shampoo 110 may comprise the ingredients shown in Table 5 below. All percentages in Table 5 are by weight and approximate for the entire mixture, making up 100% as shown in Table 5.

TABLE 5
Moisturizing Shampoo Mixture Example

| INCI Name | Purpose | Percentage |
|---|---|---|
| Water | | 56.25 |
| Guar | Active - Hair | 0.80 |
| | Conditioning | |
| Hydroxypropyltrimonium Chloride | | |
| Citric Acid | pH Adjuster | 0.05 |
| Cocamidopropyl Hydroxysultaine | Cleansing | 18.00 |
| | Surfactant | |
| Sodium Cocoyl Isethionate | Cleansing | 3.50 |
| | Surfactant | |
| Sodium Lauroyl | Cleansing | 5.00 |
| Methyl Isethionate | Surfactant | |
| Glycol Distearate | Pearlizer | 1.00 |
| Polyquaternium-73 | Active - Hair | 1.20 |
| | Conditioning | |
| Orbignya Oleifera Seed Oil | Emollient | 0.10 |
| Aloe Barbadensis Leaf Juice | Active | 0.10 |
| Phenoxyethanol (and) Caprylyl Glycol | Preservative | 1.00 |
| (and) Ethylhexylglycerin (and) Hexylene | | |
| Glycol | | |
| Water (and) Hydrolyzed Wheat Protein | Active Hair | 1.00 |
| (and) Hydrolyzed Soy Protein (and) | Conditioning | |
| Hydrolyzed Corn Protein | | |
| Decyl Glucoside | Cleansing | 2.50 |
| | Surfactant | |
| Rosa Damascena Flower Extract | Fragrance | 0.90 |
| (and) Bulnesia Sarmientoi Wood Oil | | |
| (and) Ormenis Multicaulis (Chamomile) | | |
| Oil (and) Eugenia Caryophyllus | | |
| (Clove) Leaf Oil (and) Cymbopogon Martini | | |
| Oil (and) Fusanus Spicatus (Sandal Wood) | | |
| Wood Oil Water | | |
| Acrylates Crosspolymer-4 | Thickener | 2.50 |
| Tetrasodium Glutamate | Chelator | 0.20 |
| Sodium Hydroxide | pH Adjuster | 0.40 |
| Citric Acid | pH Adjuster | 0.50 |
| | | 100% |

### Conditioning

Again, referring to FIG. 1, the conditioning mixture 112 of the system 100 may combine protein and moisture to penetrate the hair shaft to both strengthen and moisturize the newly reformed hair. In one embodiment, the conditioning mixture 112 may strengthen the hair using proteins. For example as shown in Table 6 below, hydrolyzed wheat, hydrolyzed soy and hydrolyzed corn proteins may be used; however, other similar proteins may be used. In one embodiment, the conditioning mixture 112 may add moisture and smoothness to the hair using natural oils rather than silicones, sulfates, or parabens. Such natural oils may include jojoba, avocado, sunflower seed, and bran, as shown in Table 6 below; however, other similar natural oils may be used. Additionally, in one embodiment, the conditioning mixture 112 may have a lightweight formulation that adds bounce and body to the hair. The conditioning mixture may also work together with the neutralizing shampoo mixture 108 and the moisturizing shampoo mixture 110 to further restore, replenish and strength the hair, and may include a pH adjuster to assist the neutralizing shampoo mixture 108 and the moisturizing shampoo mixture 110 in returning the hair to normal pH levels.

In one embodiment, penetration of the conditioning mixture 112 into the hair may be increased by the application of heat. For example, by applying heat to the conditioning mixture 112 while it is on the hair, the conditioning mixture 112 may activate to further fortify and penetrate the hair. Heat may be applied for 30 minutes or less using steam (e.g., via a steamer), a hooded dryer, or the like.

In one embodiment, the conditioning mixture 112 may comprise the ingredients shown in Table 6 below. All percentages in Table 6 are approximate by weight for the entire mixture, making up 100% as shown in Table 6.

TABLE 6
Conditioning Mixture Example

| INCI Name | Purpose | Percentage |
|---|---|---|
| Water | | 77.85 |
| Glycerin | Humectant | 4.00 |
| Tetrasodium Glutamate | Chelator | 0.20 |
| Diacetate | | |
| Guar | Active - Hair | 0.60 |
| | Conditioning | |
| Hydroxypropyltrimonium | | |
| Chloride | | |
| Citric Acid | pH Adjuster | 0.25 |
| Behentrimonium Chloride | Active - Hair | 3.00 |
| | Conditioning | |
| Cetearyl Alcohol | Thickener | 5.50 |
| Simmondsia Chinensis | Emollient | 1.50 |
| (Jojoba) Seed Oil | | |
| Persea Gratissima | Emollient | 1.50 |
| (Avocado) Oil | | |
| Oryza Sativa (Rice) Bran | Antioxidant | 0.10 |
| Extract (and) Rosmarinus Officinalis | | |
| (Rosemary) Extract (and) Helianthus Annuus | | |
| (Sunflower) Extract (and) Tocopherol | | |
| Polyquaternium-37 (and) Dicaprylyl Carbonate | Active - Hair | 2.50 |
| (and) Lauryl Glucoside | Conditioning | |
| Water (and) Hydrolyzed Wheat Protein (and) | Active - Hair | 1.00 |
| Hydrolyzed Soy Protein (and) Hydrolyzed Corn | Conditioning | |
| Protein | | |
| Sodium Benzoate (and) Potassium Sorbate (and) | Preservative | 1.00 |
| Water | | |
| Rosa Damascena Flower Extract | Fragrance | 1.00 |
| (and) Thuja Occidentalis (White | | |
| Cedar) Leaf Oil (and) Ormenis Multicaulis | | |
| (Chamomile) Oil (and) Eugenia Caryophyllus | | |
| (Clove) Leaf Oil (and) Cymbopogon Martini | | |
| Oil (and) Pelargonium Graveolens | | |
| Flower (Geranium) Oil (and) Fusanus Spicatus Wood | | |
| (Sandal Wood) Oil | | |
| | | 100% |

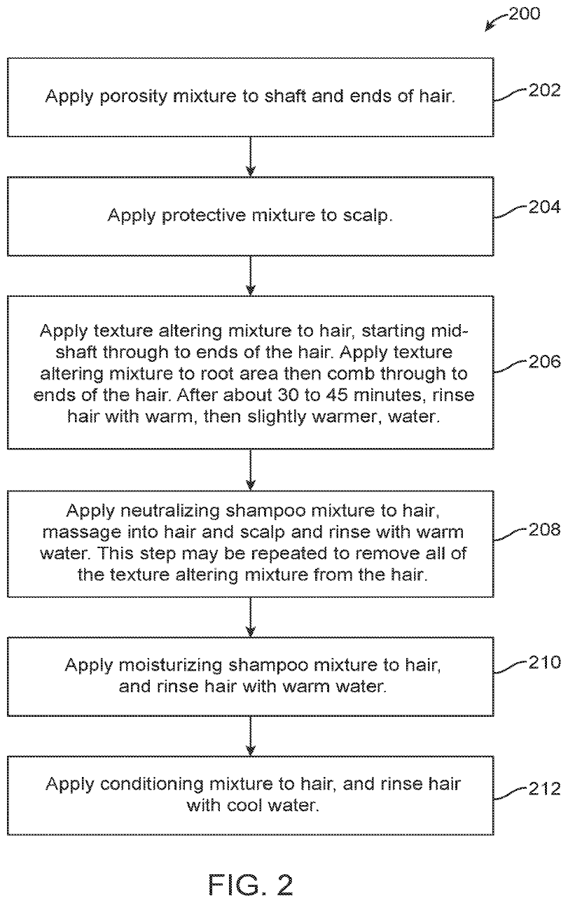

Turning now to FIG. 2, an exemplary method 200 for altering the texture of hair consistent with one or more embodiments of the present disclosure is shown. At step 202, the porosity mixture 102 may be applied to the hair shaft and the ends. As discussed above, the porosity mixture 102 prepares the hair for application of the texture altering mixture 106 by coating the hair with protein and amino acids, or the like, to fill in any pores or open spaces that may exist in the hair cuticle. This coating provides for even distribution and absorption of the texture altering mixture 106, as overly porous hair may absorb the texture altering mixture 106 faster and more unevenly than desired. The porosity mixture 102 may be rinsed from the hair.

At step 204, the protective mixture 104 may be applied to the scalp. As discussed above, the protective mixture 104 protects the scalp during application of the texture altering mixture 106 by creating a barrier between the skin and the texture altering mixture 106, thereby reducing the possibility of skin irritation.

At step 206, the texture altering mixture 106 may be applied to the hair starting at the mid-shaft to the ends of the hair. The texture altering mixture may also be applied to the root areas of the hair and combed through to the ends of the hair. After approximately 30 to 45 minutes, depending upon the amount of desired reforming of the curl pattern, the hair may be rinsed with warm (e.g., approximately 32° C. to 43° C.) and then slightly warmer water to remove the texture altering mixture 106 and the protective mixture 104 from the hair. As previously discussed, without actually straightening the hair, the texture altering mixture 106 breaks the disulfide bonds of the hair causing the natural curl pattern of the hair shaft to reform into a less curly or wavy pattern.

At step 208, the neutralizing shampoo mixture 108 may be massaged into the hair and scalp, and rinsed from the hair with warm (e.g., approximately 32° C. to 43° C.) water. This step may be repeated, as needed, to remove all of the texture altering mixture 106 from the hair. As discussed above, the neutralizing shampoo mixture 108 may be applied to the hair and massaged into the hair and scalp to gently cleanse the hair, to add moisture to the hair, and to lower the pH of the hair to normal levels. As further discussed above, the neutralizing shampoo mixture 108 may include an indicator (e.g., color indicator, or the like) that lets a stylist or another know when the texture altering mixture 106 is completely removed from the hair. For example, after rinsing the texture altering mixture 106 from the hair, the neutralizing shampoo mixture 108 may be applied to the hair and worked into a lather. In one embodiment, a color indicator may be activated when residual texture altering mixture 106 is detected in the hair, which may cause the neutralizing shampoo mixture 108 lather to turn a particular color (e.g., pink, etc).

At step 210, the moisturizing shampoo mixture 110 may be applied to the hair, as discussed above, to add moisture back to the hair, leaving it hydrated and shiny without the use of silicones, sulfates, or parabens. The moisturizing shampoo mixture 110 may then be rinsed from the hair using warm water.

At step 212, as discussed above, the conditioning mixture 112 combines protein and moisture, using natural oils rather than silicones, sulfates, or parabens, to penetrate the hair shaft to both strengthen and moisturize the newly reformed hair. Once applied to the hair, the conditioning mixture 112 may be rinsed from the hair with cool water to seal the hair cuticle. In one embodiment, as discussed above, heat may be applied to the hair for 30 minutes or less, which may cause the conditioning mixture 112 to further penetrate the hair shaft to fortify, strength, and moisturize the hair.

All references throughout this application, for example, patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application. For example, a reference that is partially inconsistent is incorporated by reference except for the partially inconsistent portion of the reference.

Portions of the disclosure of this patent document may contain material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Any element in a claim that does not explicitly state “means for” performing a specified function, or “step for” performing a specified function, is not to be interpreted as a “means” or “step” clause as specified in 35 U.S.C. § 112 ¶6. In particular, any use of “step of” in the claims is not intended to invoke the provision of 35 U.S.C. § 112 ¶6.

Persons of ordinary skill in the art may appreciate that numerous configurations may be possible to enjoy the functional benefits of the inventive systems and methods. Thus, given the wide variety of configurations and arrangements of embodiments of the present disclosure the scope of the invention is reflected by the breadth of the claims below rather than narrowed by the embodiments described above.

I claim:

1. A hair texture altering system, the hair texturing system comprising:
a. a texture altering mixture, wherein the texture altering mixture is formulated to reform, but not straighten, a curl pattern of hair;
b. a protective mixture, wherein the protective mixture is formulated to form a barrier between the texture altering mixture and a scalp portion of a head located at a root of the hair;
c. a porosity mixture, wherein the porosity mixture is formulated to prepare the hair for receiving the texture altering mixture;
d. a neutralizing shampoo mixture, wherein the neutralizing shampoo mixture is formulated to neutralize the texture altering mixture and add moisture to the hair;
e. a moisturizing shampoo mixture, wherein the moisturizing shampoo mixture is formulated to moisturize the hair; and
f. a conditioning mixture, wherein the conditioning mixture is configured to improve a condition of the hair.

2. The hair texture altering system of claim 1, wherein the curl pattern of the hair is selected from a group consisting of curly, super curly, or wavy.

3. The hair texture altering system of claim 1, wherein the texture altering mixture adds moisture to the hair.

4. The hair texture altering system of claim 3, wherein the texture altering mixture comprises one or more natural oils to add moisture to the hair.

5. The hair texture altering system of claim 4, wherein one of the one or more natural oils comprises about 0.10% by weight simmondsia chinensis (jojoba) seed oil.

6. The hair texture altering system of claim 4, wherein one of the one or more natural oils comprises about 0.10% by weight helianthus annuus (sunflower) seed oil.

7. The hair texture altering system of claim 1, wherein the texture altering mixture is free of parabens, sulfates, silicones, synthetic fragrance, mineral oil, petrolatum, and synthetic petrochemicals.

8. The hair texture altering system of claim 1, wherein the texture altering mixture comprises about 1.35% by weight sodium hydroxide.

9. The hair texture altering system of claim 1, wherein the texture altering mixture comprises about 1.48% by weight of sodium hydroxide.

10. The hair texture altering system of claim 1, wherein the texture altering mixture comprises about 1.35% to 1.75% by weight sodium hydroxide.

11. The hair texture altering system of claim 1, wherein the protective mixture comprises one or more natural oils.

12. The hair texture altering system of claim 11, wherein the protective mixture comprises a base, wherein the base comprises at least one of the one or more natural oils.

13. The hair texture altering system of claim 12, wherein the at least one of the one or more natural oils of the base comprises about 55.0% by weight helianthus annuus (sunflower) seed oil.

14. The hair texture altering system of claim 11, wherein at least one of the one or more natural oils comprises simmondsia chinensis (jojoba) seed oil.

15. The hair texture altering system of claim 1, wherein the protective mixture comprises curcuma longa (turmeric) root extract.

16. The hair texture altering system of claim 1, wherein a consistency of the protective mixture is a gel-like oil.

17. The hair texture altering system of claim 1, wherein the protective mixture is free of parabens, sulfates, silicones, synthetic fragrance, mineral oil, petrolatum, and synthetic petrochemicals.

18. The hair texture altering system of claim 1, wherein preparing the hair for receiving the texture altering mixture comprises coating the hair with proteins and/or amino acids for even distribution and absorption of the texture altering mixture on the hair.

19. The hair texture altering system of claim 1, wherein the neutralizing shampoo mixture comprises about 0.10% by weight of aloe barbadensis leaf juice to add moisture to the hair.

20. The hair texture altering system of claim 1, wherein the neutralizing shampoo mixture is free of parabens, sulfates, silicones, synthetic fragrance, mineral oil, petrolatum, and synthetic petrochemicals.

21. The hair texture altering system of claim 1, wherein the moisturizing shampoo mixture comprises about 0.10% by weight of aloe barbadensis leaf juice to moisturize the hair.

22. The hair texture altering system of claim 1, wherein improving the condition of the hair comprises moisturizing the hair using one or more natural oils, and wherein at least one of the one or more natural oils is selected from a group consisting of: simmondsia chinensis (jojoba) seed oil and persea gratissima (avocado) oil, guar hydroxypropyltrimonium chloride, and Oryza sativa (rice) bran extract.

23. The hair texture altering system of claim 22, wherein the conditioning mixture comprises about 1.50% by weight of simmondsia chinensis (jojoba) seed oil.

24. The hair texture altering system of claim 22, wherein the conditioning mixture comprises about 1.50% by weight of the persea gratissima (avocado) oil.

25. The hair texture altering system of claim 1, wherein improving the condition of the hair comprises strengthening the hair, wherein the conditioning mixture comprises one or more proteins to strengthen the hair, and wherein at least one of the one or more proteins is selected from a group consisting of hydrolyzed wheat protein, hydrolyzed soy protein, or hydrolyzed corn protein.

26. The hair texture altering system of claim 1, wherein the conditioning mixture is free of parabens, sulfates, silicones, synthetic fragrance, mineral oil, petrolatum, and synthetic petrochemicals.

27. A method of altering hair texture comprising the steps of:
a. applying a texture altering mixture to hair, wherein the texture altering mixture is formulated to reform, but not straighten, a curl pattern of hair;
b. applying a protective mixture, wherein the protective mixture is formulated to create a barrier between the texture altering mixture and a scalp portion of a head located at the root of the hair;
c. applying a porosity mixture to the hair, wherein the porosity mixture is formulated to prepare the hair for application of the texture altering mixture;
d. applying a neutralizing shampoo mixture to the hair, wherein the neutralizing shampoo mixture is formulated to neutralize the texture altering mixture and add moisture to the hair;
e. applying a moisturizing shampoo mixture to the hair, wherein the moisturizing shampoo mixture is formulated to moisturize the hair; and
f. applying a conditioning mixture, wherein the conditioning mixture is configured to improve the condition of the hair, and wherein improving the condition of the hair comprises moisturizing and strengthening the hair.

28. The method of altering hair texture of claim 27, wherein the texture altering mixture comprising about 1.35% by weight sodium hydroxide.

29. The method of altering hair texture of claim 27, wherein the texture altering mixture comprises about 1.48% by weight sodium hydroxide. 20
